(19) United States
(12) Patent Application Publication
(10) Pub. No.: US 2011/0288375 A1
(43) Pub. Date: Nov. 24, 2011

(54) ENDOSCOPE

(75) Inventor: Naoya OUCHI, Tokyo (JP)

(73) Assignee: OLYMPUS MEDICAL SYSTEMS CORP., Tokyo (JP)

(21) Appl. No.: 13/090,407

(22) Filed: Apr. 20, 2011

Related U.S. Application Data
(63) Continuation of application No. PCT/JP2010/069828, filed on Nov. 8, 2010.

(30) Foreign Application Priority Data
Mar. 12, 2010 (JP) 2010-056425

(51) Int. Cl. A61B 1/01

(52) U.S. Cl. 600/114

(57) ABSTRACT

An endoscope is provided that includes: an insertion portion; a bending portion having an independently bendable first bending region, and a second bending region that is bendable in the same direction as a bending direction of the first bending region together with the first bending region; a flexible tube portion; a first wire that, when pulled, causes only the first bending region to bend; a first guide pipe through which the first wire is inserted, and which has a distal end that is fixed to an intermediate pipe sleeve; a second wire that, when pulled, causes the second bending region to bend in conjunction with bending of the first bending region; and a second guide pipe through which the second wire is inserted, and which has one part that is fixed to a connecting pipe sleeve, and has a distal end that is positioned inside the second bending region in a condition in which the distal end protrudes forward by a first length from a distal end of the flexible tube portion in a non-fixed state.

### CROSS REFERENCE TO RELATED APPLICATION

This application is a continuation application of PCT/JP2010/069828 filed on Nov. 8, 2010 and claims benefit of Japanese Application No. 2010-056425 filed in Japan on Mar. 12, 2010, the entire contents of which are incorporated herein by this reference.

### BACKGROUND OF THE INVENTION

1. Field of the Invention

The present invention relates to an endoscope that has an elongated insertion portion that is inserted into a subject, and a bending portion that is provided in the insertion portion and includes a first bending region and a second bending region.

2. Description of the Related Art

In recent years, endoscopes have been widely used in the medical field and industrial field. An endoscope used in the medical field observes an organ inside a body cavity of a subject by inserting an elongated insertion portion into the body cavity. As necessary, the endoscope can perform various kinds of treatment using a treatment instrument that is inserted inside a treatment instrument insertion channel provided in the endoscope.

Further, with an endoscope used in the industrial field, by inserting an elongated insertion portion of the endoscope inside an object such as a jet engine or pipes of a factory, it is possible to conduct an observation to check for the presence of flaws or corrosion or the like at a site to be examined inside the object and to perform various kinds of repairs.

A configuration is known in which a bending portion that is bendable in a plurality of directions is provided in an insertion portion of an endoscope. The bending portion improves the advancing ability of the insertion portion at a curved section inside a duct, and also varies an observation direction of an observation optical system provided in a distal end portion that is positioned further on a distal end side in the insertion direction (hereunder, referred to simply as “distal end side”) than the bending portion in the insertion portion.

Normally, a bending portion provided in an insertion portion of an endoscope is configured to be bendable in, for example, four directions, that is, upward, downward, leftward, and rightward, by connecting a plurality of bending pieces along the insertion direction of the insertion portion.

Further, the bending portion is configured to be bendable in any of the upward, downward, leftward, and rightward directions by performing a pulling operation from an operation portion with respect to any of four wires that are passed through the inside of the insertion portion and whose distal ends are fixed to a bending piece that is positioned furthest on the distal end side among the bending pieces.

In this connection, as described above, the bending portion is bent with a certain bending radius that takes a proximal end side in the insertion direction (hereunder, referred to simply as “proximal end side”) of the bending portion as a starting point when an operation to pull a wire is performed. However, depending on the internal shape of the subject or object into which an insertion portion is inserted, a configuration has been desired that can freely vary the bending radius of a bending portion to improve the insertability of the insertion portion inside a subject or object.

Therefore, Japanese Patent Application Laid-Open Publication No. 2006-320501 discloses a configuration of an endoscope in which a first bending portion and a second bending portion are provided as two bending portions on a distal end side of an insertion portion of an endoscope. The first bending portion and the second bending portion are independently bendable, and therefore the bending radius of the bending portion can be freely varied.

More specifically, FIG. 9 is a partial cross-sectional view that shows the configuration of the bending portion of the insertion portion of the conventional endoscope as well as a distal end side of a flexible tube portion, FIG. 10 is a cross-sectional view of the first bending portion along a line X-X in FIG. 9, FIG. 11 is a cross-sectional view of the second bending portion along a line XI-XI in FIG. 9, and FIG. 12 is a cross-sectional view of a connecting pipe sleeve along a line XII-XII in FIG. 9.

As shown in FIG. 9, a bending portion 100 includes, as principal components, a first bending portion 101 that is positioned on a distal end side, a second bending portion 103 that is positioned on a proximal end side of the first bending portion 101, and an intermediate pipe sleeve 102 that is provided between the first bending portion 101 and the second bending portion 103. A distal end side of a flexible tube portion 105 is connected through a connecting pipe sleeve 104 to a proximal end side of the second bending portion 103.

A plurality of bending pieces 101k are connected along an insertion direction S inside the first bending portion 101. A plurality of bending pieces 103k are also connected along the insertion direction S inside the second bending portion 103. The outer circumferences of the bending pieces 101k and 103k are covered with a braid 110, and the outer circumference of the braid 110 is covered with a bending rubber 111.

As shown in FIG. 9 to FIG. 12, four first wires 120 that are used when bending the first bending portion 101, for example, in any of the four directions of upward, downward, leftward, and rightward, are inserted through the inside of the insertion portion in a condition in which the positions of the four first wires 120 are staggered by, for example, 90° in the circumferential direction. A distal end of each of the first wires 120 is fixed to a bending piece 101k that is located at the farthest position on the distal end side among the bending pieces 101k.

As shown in FIG. 9 and FIG. 10, each of the first wires 120 is respectively supported by wire receivers 122 that are provided in each bending piece 101k inside the first bending portion 101. Further, as shown in FIG. 9, FIG. 11, and FIG. 12, the first wires 120 are covered by first guide pipes 121 inside the second bending portion 103 and the flexible tube portion 105. In this connection, the distal ends of the first guide pipes 121 are fixed to the intermediate pipe sleeve 102.

Further, as shown in FIG. 9, FIG. 11, and FIG. 12, second wires 130 that are used when bending the second bending portion 103, for example, in the two directions of upward and downward are inserted through the inside of the insertion portion, for example, in a condition in which the positions of the second wires 130 are staggered by 180° in the circumferential direction. A distal end of each of the second wires 130 is fixed to a bending piece 103k that is located at the farthest position on the distal end side among the bending pieces 103k.

Further, as shown in FIG. 9 and FIG. 11, each of the second wires 130 is respectively supported by wire receivers 132 that are provided in each bending piece 103k inside the second bending portion 103. Further, as shown in FIG. 9 and FIG. 12, the second wires 130 are covered by a second guide pipe 131 inside the flexible tube portion 105. In this connection, the distal ends of the second guide pipes 131 are fixed to the connecting pipe sleeve 104.

Hence, in the conventional endoscope, for example, when a user wishes to bend only the first bending portion 101, among the four first wires 120, the user pulls the first wire 120 that is positioned in a direction in which the user wishes to bend the first bending portion 101, and as a result the first bending portion 101 bends in a manner that takes the distal end of the first guide pipe 121 as a starting point. Further, when the user wishes to bend only the second bending portion 103, among the two second wires 130, the user pulls the second wire 130 that is positioned in a direction in which the user wishes to bend the second bending portion 103, and as a result the second bending portion 103 bends in a manner that takes the distal end of the second guide pipe 131 as a starting point.

### SUMMARY OF THE INVENTION

An endoscope according to one aspect of the present invention includes: an elongated insertion portion that is inserted inside a subject; a bending portion that is provided in the insertion portion, and that has an independently bendable first bending region, and a second bending region that is connected in series to a proximal end side of the first bending region and that is bendable in a same direction as a bending direction of the first bending region together with the first bending region; a flexible tube portion that is connected in series to a proximal end side of the bending portion in the insertion portion; a first wire which is inserted through an inside of the insertion portion and which has a distal end that is fixed to a distal end of the first bending region, and which causes only the first bending region to bend when the first wire is pulled; a first guide pipe, through an inside of which the first wire is inserted to freely advance and retract in an insertion direction of the insertion portion, and which has a distal end that is fixed between the first bending region and the second bending region; a second wire which is inserted through the inside of the insertion portion and which has a distal end that is fixed to the distal end of the first bending region, and which causes the second bending region to bend in conjunction with bending of the first bending region when the second wire is pulled; and a second guide pipe, through an inside of which the second wire is inserted to freely advance and retract in the insertion direction, one part of the second guide pipe being fixed between the second bending region and the flexible tube portion, and which has a distal end that protrudes forward in the insertion direction by a first length from a distal end of the flexible tube portion and is positioned inside the second bending region in a non-fixed state.

### BRIEF DESCRIPTION OF THE DRAWINGS

FIG. 1 is a perspective view that illustrates the external appearance of an endoscope according to the present embodiment;

FIG. 2 is a cross-sectional view of an insertion portion along a line II-II in FIG. 1;

FIG. 3 is a cross-sectional view of a first bending region along a line III-III in FIG. 2;

FIG. 4 is a cross-sectional view of a second bending region along a line IV-IV in FIG. 2;

FIG. 5 is an enlarged view of a region surrounded by an alternate long and short dash line V of the insertion portion in FIG. 1;

FIG. 6 is a view that shows a state in which only the first bending region shown in FIG. 5 is bent accompanying pulling of a first wire;

FIG. 7 is a view that shows a state in which, accompanying pulling of a second wire, the second bending region is bent in the same direction as the first bending region shown in FIG. 5 together with the first bending region;

FIG. 8 is a view that shows a conventional insertion portion in which a second bending region bends in a different direction to a first bending region accompanying pulling of a first wire;

FIG. 9 is a partial cross-sectional view that shows the configuration of a bending portion of an insertion portion of a conventional endoscope as well as a distal end side of a flexible tube portion;

FIG. 10 is a cross-sectional view of a first bending portion along a line X-X in FIG. 9;

FIG. 11 is a cross-sectional view of a second bending portion along a line XI-XI in FIG. 9; and

FIG. 12 is a cross-sectional view of a connecting pipe sleeve along a line XII-XII in FIG. 9.

### DETAILED DESCRIPTION OF THE PREFERRED EMBODIMENT

An embodiment of the present invention is described hereunder with reference to the drawings. It should be noted that the drawings are schematic ones in which the relationship between the thickness and width of each member, the thickness ratios of the members, and the like are different from those of actual members. Naturally, the drawings include portions in which the dimensional relationships and ratios are different from one another.

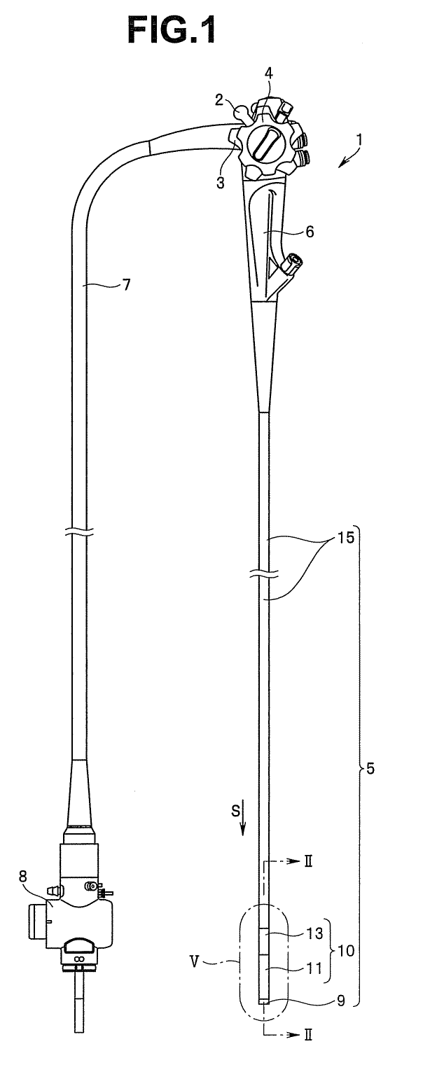

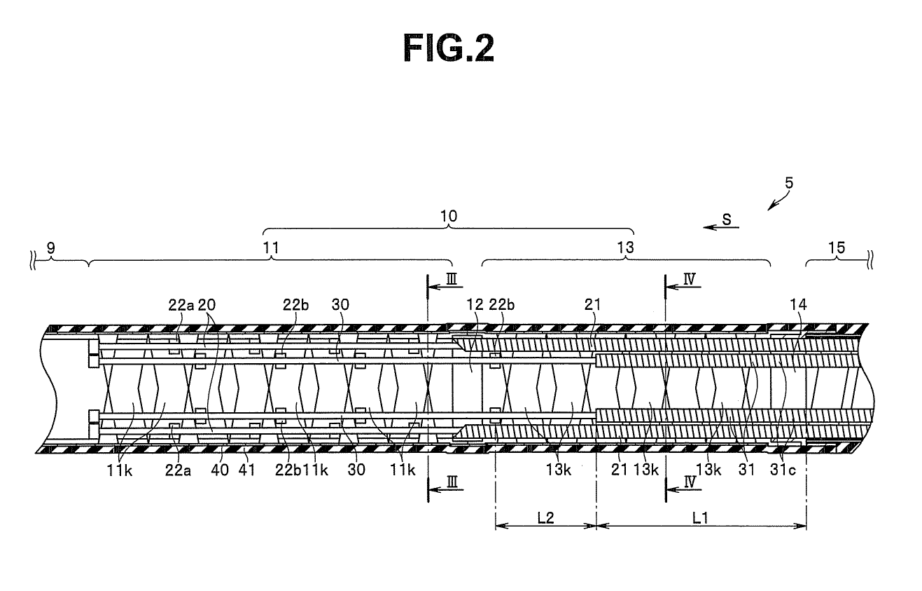

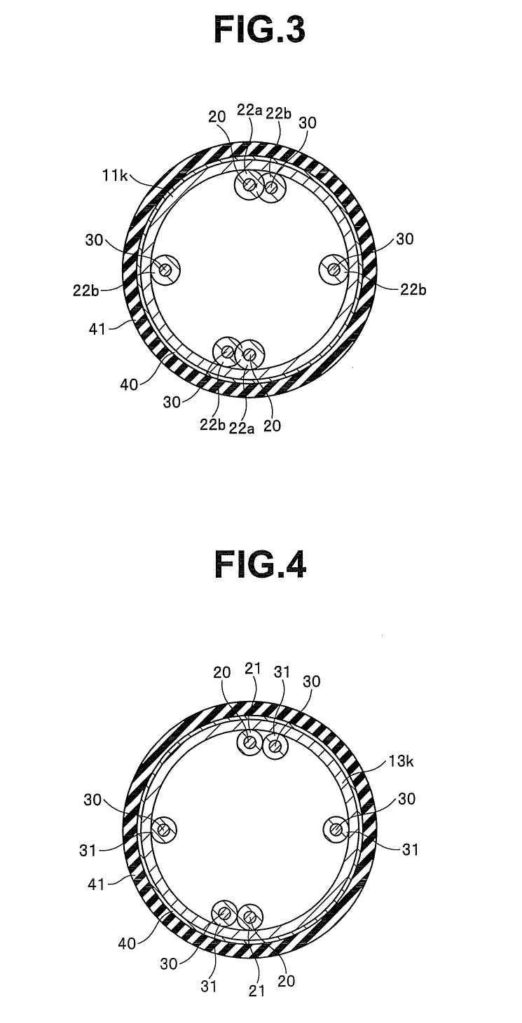

FIG. 1 is a perspective view that illustrates the external appearance of an endoscope according to the present embodiment. FIG. 2 is a cross-sectional view of an insertion portion along a line II-II in FIG. 1. FIG. 3 is a cross-sectional view of a first bending region along a line III-III in FIG. 2. FIG. 4 is a cross-sectional view of a second bending region along a line IV-IV in FIG. 2.

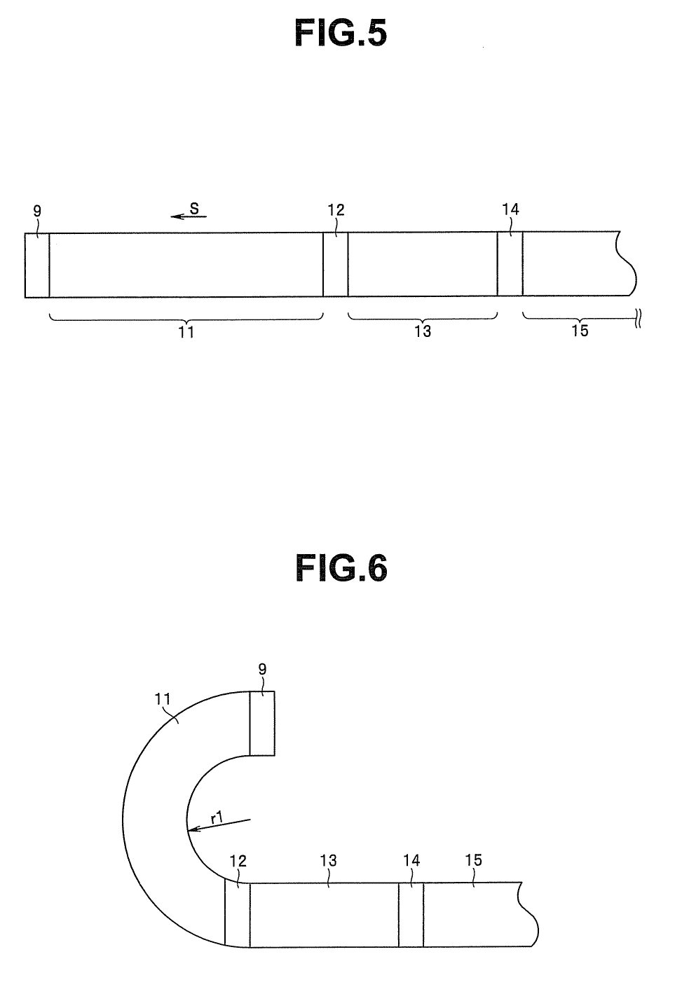

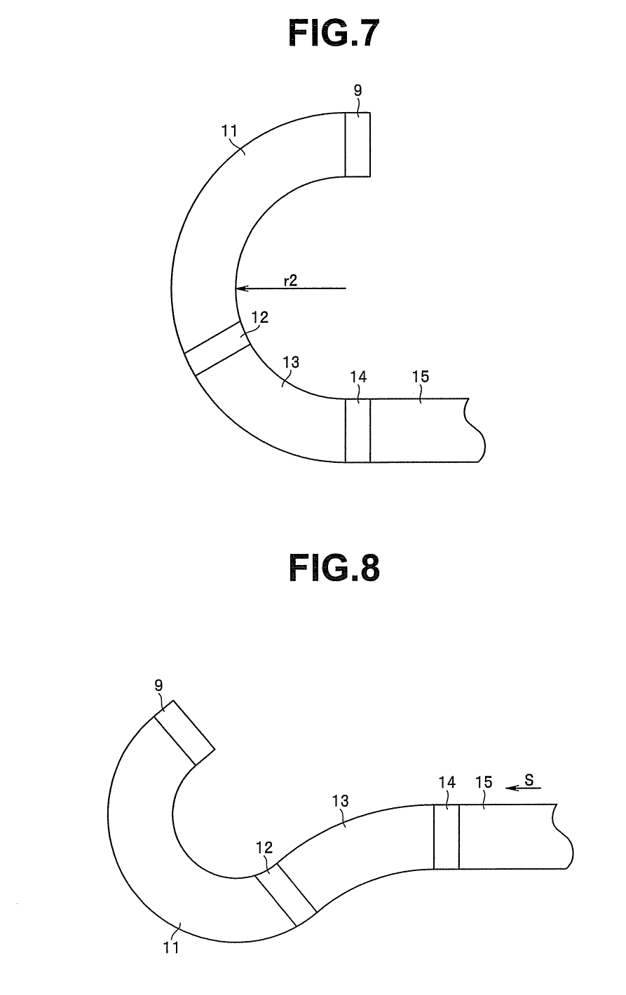

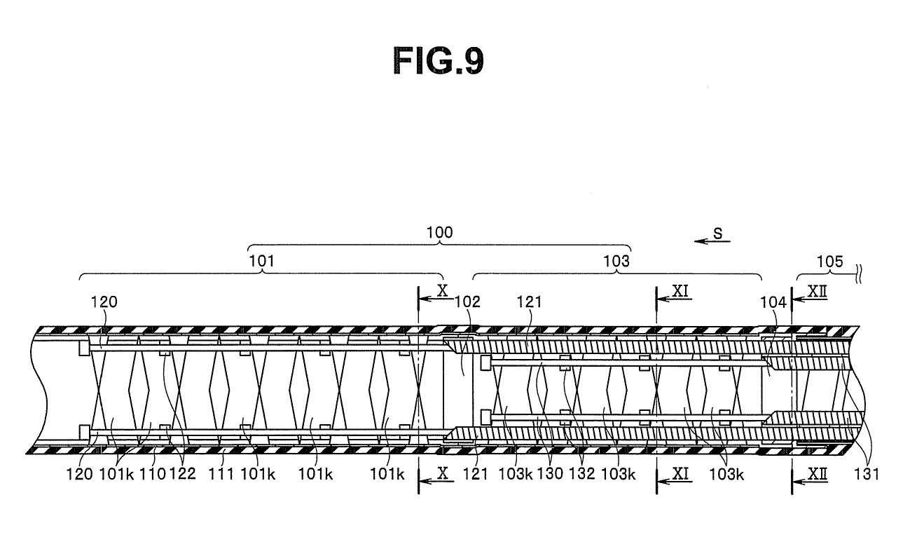

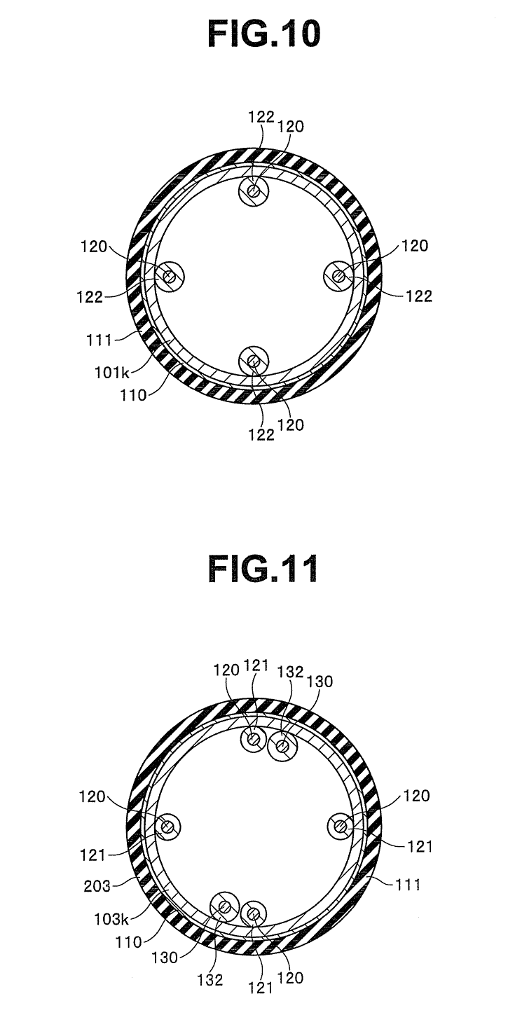

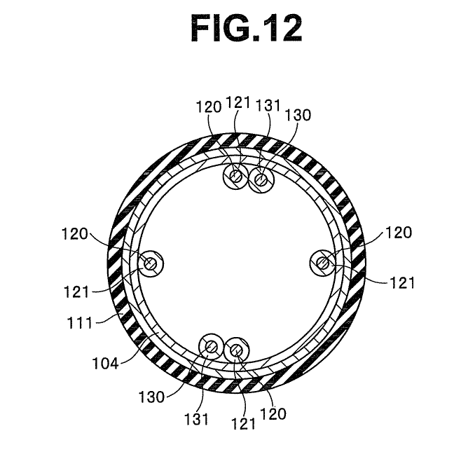

FIG. 5 is an enlarged view of a region surrounded by an alternate long and short dash line V of the insertion portion in FIG. 1. FIG. 6 is a view that shows a state in which only the first bending region shown in FIG. 5 is bent accompanying pulling of a first wire. FIG. 7 is a view that shows a state in which, accompanying pulling of a second wire, the second bending region is bent in the same direction as the first bending region shown in FIG. 5 together with the first bending region. FIG. 8 is a view that shows a conventional insertion portion in which a second bending region bends in a different direction to a first bending region accompanying pulling of a first wire.

As shown in FIG. 1, an endoscope 1 includes, as principal components, an insertion portion 5 that is inserted into a subject, an operation portion 6 that is connected in series to a proximal end side in an insertion direction S of the insertion portion 5, a universal cord 7 that extends from the operation portion 6, and a connector 8 that is provided at an extending end of the universal cord 7. The endoscope 1 is electrically connected to an external apparatus such as a control apparatus or an illumination apparatus through the connector 8.

A bending operation lever for the first bending region (hereunder, referred to simply as “lever”) 2, an upward/downward bending operation knob for the second bending region (hereunder, referred to simply as “knob”) 3, and a leftward/rightward bending operation knob for the second bending region (hereunder, referred to simply as “knob”) 4 are provided in the operation portion 6.

The insertion portion 5 includes a distal end portion 9, a bending portion 10, and a flexible tube portion 15, and is formed in an elongated shape along an insertion direction S.

An unshown image pickup unit that observes the inside of a subject and an illumination unit that illuminates the inside of a subject and the like are provided inside the distal end portion 9.

The bending portion 10 includes a first bending region 11 that is independently bendable in, for example, two directions, that is, upward and downward, by an operation of the lever 2, and a second bending region 13 that, by an operation of the knob 3 or the knob 4, is bendable in the same direction as the bending direction of the first bending region 11 together with the first bending region 11 in, for example, four directions, that is, upward, downward, leftward, and rightward.

As shown in FIG. 2 and FIG. 5, an intermediate pipe sleeve 12 is provided between the first bending region 11 and the second bending region 13. The first bending region 11 may have a configuration that is independently bendable in the two directions of leftward and rightward, and not the two directions of upward and downward.

The flexible tube portion 15 is connected in series to the proximal end side of the bending portion 10, that is, the proximal end side of the second bending region 13. As shown in FIG. 2, the flexible tube portion 15 is connected in series to the proximal end side of the second bending region 13 through a connecting pipe sleeve 14.

Further, as shown in FIG. 2, a plurality of bending pieces 11k that are bendable in four directions are provided in a linked manner along the insertion direction S inside the first bending region 11. Furthermore, a plurality of bending pieces 13k that are bendable in four directions are also provided in a linked manner along the insertion direction S inside the second bending region 13.

As shown in FIG. 2 to FIG. 4, the outer circumferences of the plurality of bending pieces 11k and 13k are covered with a braid 40, and the outer circumference of the braid 40 is covered with a bending rubber 41.

Further, as shown in FIG. 2 to FIG. 4, two first wires 20 that cause the first bending region 11 to independently bend in, for example, the two directions of upward and downward are inserted through the inside of the insertion portion 5 so as to be located at positions that are different from each other by, for example, 180° in the circumferential direction.

Inside the bending pieces 11k of the first bending region 11, the positions of the first wires 20 in the circumferential direction are regulated and supported by wire receivers 22a that are provided in each bending piece 11k. Further, as shown in FIG. 2, the distal ends of the first wires 20 are fixed to a bending piece 11k that is located at the farthest position on the distal end side in the insertion direction S among the plurality of bending pieces 11k. In this connection, the proximal end of each first wire 20 is wound around an unshown pulley that can be rotated by the lever 2 that is provided inside the operation portion 6.

As shown in FIG. 2 and FIG. 4, inside the bending pieces 13k of the second bending region 13, the outer circumference of each first wire 20 is covered with a first guide pipe 21, respectively, that has a distal end that is fixed to the intermediate pipe sleeve 12. The first wires 20 are inserted through the inside of the corresponding first guide pipes 21 so as to freely advance and retract in the insertion direction S.

The first guide pipes 21 regulate the positions in the circumferential direction of the first wires 20 inside the second bending region 13, and also prevent the first wires 20 from contacting a built-in member of the second bending region 13.

Because the distal end of the first guide pipe 21 is fixed to the intermediate pipe sleeve 12, when either one of the two first wires 20 for upward/downward bending is pulled by means of an operation of the lever 2, as shown in FIG. 6, the first bending region 11 independently bends in either the upward direction or downward direction in a manner that takes the distal end of the first guide pipe 21 as a starting point. More specifically, as shown in FIG. 6, the bending portion 10 bends with a bending radius r1.

As shown in FIG. 2 to FIG. 4, four second wires 30 that cause the second bending region 13 to bend in four directions, that is, for example, upward, downward, leftward, and rightward in conjunction with bending of the first bending region 11 are inserted through the inside of the insertion portion 5 so as to be located at positions that are different from each other by, for example, 90° in the circumferential direction.

Note that to facilitate understanding of the drawing, the illustration of two of the second wires 30 among the four second wires 30 is omitted from FIG. 2, and although the illustration in FIG. 2 shows that two of the second wires 30 are located at positions that are further on the inside in the diametrical direction than the two first wires 20, actually, as shown in FIG. 3 and FIG. 4, the two second wires 30 are located at positions that are side by side with the two first wires 20 in the circumferential direction.

Inside the bending pieces 11k of the first bending region 11, the positions of the second wires 30 in the circumferential direction are regulated and supported by wire receivers 22b that are provided in each bending piece 11k. As shown in FIG. 2, the distal ends of the second wires 30 are fixed to a bending piece 11k that is located at the farthest position on the distal end side in the insertion direction S among the plurality of bending pieces 11k.

In this connection, the proximal end of each of the two second wires 30 for upward/downward bending is wound around an unshown pulley that can be rotated by the knob 3 that is provided inside the operation portion 6, and the proximal end of each of the two second wires 30 for leftward/rightward bending is wound around an unshown pulley that can be rotated by the knob 4 that is provided inside the operation portion 6.

Further, as shown in FIG. 2 and FIG. 4, inside the bending pieces 13k of the second bending region 13, the outer circumference of each second wire 30 is covered with a second guide pipe 31, respectively. A midway position 31c on a distal end side of each second guide pipe 31 is fixed to the connecting pipe sleeve 14. In this connection, the second wires 30 are inserted through the inside of the second guide pipes 31, respectively, so as to freely advance and retract in the insertion direction S.

As shown in FIG. 2, the distal end of each second guide pipe 31 protrudes forward in the insertion direction S by a first length L1 from the distal end of the flexible tube portion 15 and is positioned inside the second bending region 13. Further, the distal end of each second guide pipe 31 is positioned to the rear in the insertion direction S by a second length L2 from a wire receiver 22b that is located at a position that is furthest to the rear in the insertion direction S among the wire receivers 22b, and thus the distal ends of the second guide pipes 31 are in a non-fixed state.

More specifically, the respective second guide pipes 31 do not cover the outer circumference of the entire region of the corresponding second wire 30 located inside the second bending region 13, but rather cover only the outer circumference of the region of the second wire 30 that protrudes forward by the first length L1 from the distal end of the flexible tube portion 15. In other words, inside the second bending region 13, the second guide pipes 31 do not cover the outer circumference of the region of the corresponding second wires 30 that extends from the wire receiver 22b that is located at the most rearward position as far as the rear of the second length L2.

The second guide pipes 31 regulate the positions in the circumferential direction of the second wires 30 inside the second bending region 13, and also prevent the second wires 30 from contacting a built-in member of the second bending region 13.

The distal end of each second wire 30 is fixed to a bending piece 11k that is located at the farthest position on the distal end side, and the midway position 31c on the distal end side of the second guide pipe 31 is fixed to the connecting pipe sleeve 14.

Therefore, when either one of the two second wires 30 for leftward/rightward bending is pulled by an operation of the knob 4, as shown in FIG. 7, the second bending region 13 bends in the leftward direction or the rightward direction together with the first bending region 11 in a manner that takes the midway position 31c of the second guide pipe 31 as a starting point.

Alternatively, when either one of the two second wires 30 for upward/downward bending is pulled by an operation of the knob 3, as shown in FIG. 7, the second bending region 13 bends in the upward direction or the downward direction together with the first bending region 11 in a manner that takes the midway position 31c of the second guide pipe 31 as a starting point. More specifically, as shown in FIG. 7, the bending portion 10 bends with a bending radius r2 that is greater than the bending radius r1.

As described above, the second guide pipes 31 do not cover the outer circumference of the entire region of the second wires 30 that are located inside the second bending region 13. More specifically, each second guide pipe 31 covers only the first length L1, and does not cover the second length L2.

The reason is that if a configuration is adopted in which the distal end of the second guide pipe 31 is positioned in the intermediate pipe sleeve 12 and the outer circumference of the entire region of the second wire 30 that is positioned inside the second bending region 13 is covered by the second guide pipe 31, for example, when the second wire 30 for upward bending is pulled in order to bend the first bending region 11 and the second bending region 13 upward, interference would occur between the distal end of the second guide pipe 31 that covers the outer circumference of the second wire 30 for upward bending and the wire receiver 22b that is located at the most rearward position, and as a result bending in the upward direction could not be performed. To prevent such a situation, each second guide pipe 31 covers only the first length L1, and does not cover the second length L2.

Furthermore, a configuration may also be considered in which the wire receivers 22b need not be provided as long as the positions in the circumferential direction of the second wires 30 can be regulated by the second guide pipes 31 and, in a state in which the midway positions 31c are fixed to the connecting pipe sleeve 14, the distal ends of the second guide pipe 31 are not fixed and the second guide pipes 31 also cover the outer circumferences of the second wires 30 up to the distal ends thereof, that is, the outer circumferences of the second wires 30 that are positioned inside the first bending region 11.

However, since the position regulating force of the guide pipe is weaker than that of the wire receiver, the above described configuration is not preferable because there is the possibility that the second wires 30 will become misaligned in the circumferential direction inside the first bending region 11 and the second bending region 13, and the user may be unable to bend the bending portion 10 in a desired direction.

Further, it may be considered that a configuration in which the distal ends of the second guide pipes 31 are fixed to the connecting pipe sleeve 14 and the positions of the second wires 30 located inside the second bending region 13 are regulated by the wire receivers 22b also allows the second bending region 13 to bend together with the first bending region 11.

However, according to the above configuration, for example, when the first wire 20 for upward bending is pulled, the second wire 30 for upward bending that is positioned inside the second bending region 13 is pushed to the operation portion 6 side and the second wire 30 for downward bending is dragged to the distal end side. At this time, the second wire 30 for upward bending that has been pushed to the operation portion 6 side slackens. In contrast, a tensile force acts on the second wire 30 for downward bending that has been dragged to the distal end side, and a pulling force arises at the bending pieces 13k in which the wire receivers 22b are provided.

Accordingly, by conducting experiments using various prototypes, it has been found that the second bending region 13 bends downward in an opposite direction to the bending direction of the first bending portion 11. Further, as shown in FIG. 8, the bending portion has a wave-like shape in which only the second bending region 13 bends downward even when the first bending region 11 bends upward, and this shape is not preferable in terms of usage. This result is a phenomenon that could not be foreseen even by the inventor, and the inventor conducted concentrated studies to solve this problem and conceived the present invention.

In this connection, according to the present embodiment, even if the second guide pipes 31 regulate the positions in the circumferential direction of the second wires 30 by covering the second wires 30 that are positioned inside the second bending region 13, when the first wire 20 for upward bending is pulled, the bending portion does not become a wave-like shape as shown in FIG. 8. The reason is described in detail below.

First, as shown in FIG. 2, the second wires 30 are connected to the bending piece that is located at the farthest position on the distal end side. If the wire receivers 22b and the bending pieces 13k are connected, a tensile force that acts on the second wires 30 will be transmitted to the bending pieces 13k. However, in the present embodiment, since the second guide pipes 31 and the bending pieces 13k are not connected, a tensile force that acts on the second wires 30 is not transmitted to the bending pieces 13k and the second bending region 13 does not bend.

Therefore, it is preferable that, in a state in which the midway positions 31c of the second guide pipes 31 are fixed to the connecting pipe sleeve 14, the distal ends of the second guide pipes 31 are not fixed and protrude forward by the first length L1 from the distal end of the flexible tube portion 15 to be arranged within the second bending region 13 at positions that are located at a distance equivalent to the second length L2 to the rear of the wire receiver 22b that is arranged at the most rearward position among the wire receivers 22b.

According to the present embodiment, a configuration has been described in which the distal end of each of the first wires 20 is fixed to a bending piece 11k located at the most forward position in the first bending region 11, and the distal ends of the first guide pipes 21 that cover the outer circumferences of the first wires 20, respectively, are fixed to the intermediate pipe sleeve 12.

Further, in the configuration described according to the present embodiment, the distal end of each second wire 30 is fixed to the bending piece 11k located at the most forward position in the first bending region 11, the midway position 31c on the distal end side of each second guide pipe 31 that covers the outer circumference of the corresponding second wire 30 is fixed to the connecting pipe sleeve 14, and the distal end of each second guide pipe 31 protrudes forward in a non-fixed state from the distal end of the flexible tube portion 15 by the first length L1 and is located at a position that is at a distance equivalent to the second length L2 to the rear of the wire receiver 22b that is arranged at the most rearward position among the wire receivers 22b.

Therefore, by pulling the first wires 20, only the first bending region 11 can be bent with the bending radius r1 in either of two directions, that is, the upward or downward direction, in a manner that takes the distal end of the first guide pipe 21 as a starting point. Further, by pulling the second wires 30, the second bending region 13 can be bent together with the first bending region 11 with the bending radius r2 that is greater than the bending radius r1 in the same direction as the bending direction of the first bending region 11 in a manner that takes the midway position 31c of the second guide pipe 31 as a starting point.

More specifically, since the bending radius of the bending portion 10 can be varied by a single operation in which the lever 2 is operated or the knob 3 or 4 is operated, it is possible to provide the endoscope 1 that has a configuration which can freely vary the bending radius of the bending portion 10 with a single operation, and which can improve the insertability of the insertion portion 5.

A modification example is described hereunder.

The present embodiment has a configuration in which two first wires 20 whose positions are staggered by 180° in the circumferential direction and four second wires 30 whose positions are staggered by 90° in the circumferential direction are inserted inside the insertion portion 5. As a result, the first bending region 11 is bendable in two directions by means of the first wires 20, and the second bending region 13 is bendable in four directions together with the first bending region 11 by means of the second wires 30.

However, the present invention is not limited thereto, and a configuration may also be adopted in which four first wires 20 whose positions are staggered by 90° in the circumferential direction and two second wires 30 whose positions are staggered by 180° in the circumferential direction are inserted inside the insertion portion 5 so that the first bending region 11 is bendable in four directions by means of the first wires 20, and the second bending region 13 is bendable in two directions together with the first bending region 11 by means of the second wires 30.

Furthermore, a configuration may be adopted in which four first wires 20 whose positions are staggered by 90° in the circumferential direction and, similarly to the present embodiment, four second wires 30 whose positions are staggered by 90° in the circumferential direction are inserted inside the insertion portion 5 so that the first bending region 11 is bendable in four directions by means of the first wires 20, and the second bending region 13 is also bendable in four directions together with the first bending region 11 by means of the second wires 30.

What is claimed is:

1. An endoscope, comprising:
an elongated insertion portion that is inserted inside a subject;
a bending portion that is provided in the insertion portion, and that has an independently bendable first bending region, and a second bending region that is connected in series to a proximal end side of the first bending region and that is bendable in a same direction as a bending direction of the first bending region together with the first bending region;
a flexible tube portion that is connected in series to a proximal end side of the bending portion in the insertion portion;
a first wire which is inserted through an inside of the insertion portion and which has a distal end that is fixed to a distal end of the first bending region, and which causes only the first bending region to bend when the first wire is pulled;
a first guide pipe, through an inside of which the first wire is inserted to freely advance and retract in an insertion direction of the insertion portion, and which has a distal end that is fixed between the first bending region and the second bending region;
a second wire which is inserted through the inside of the insertion portion and which has a distal end that is fixed to the distal end of the first bending region, and which causes the second bending region to bend in conjunction with bending of the first bending region when the second wire is pulled; and
a second guide pipe, through an inside of which the second wire is inserted to freely advance and retract in the insertion direction, one part of the second guide pipe being fixed between the second bending region and the flexible tube portion, and which has a distal end that protrudes forward in the insertion direction by a first length from a distal end of the flexible tube portion and is positioned inside the second bending region in a non-fixed state.

2. The endoscope according to claim 1, wherein:
inside the first bending region, a position of the second wire in a circumferential direction is regulated by a wire receiver; and
the wire receiver is located at a position that is forward in the insertion direction by a second length from the distal end of the second guide pipe.

3. The endoscope according to claim 1, wherein two of the first wires and the first guide pipes are inserted through the inside of the insertion portion in a condition in which the positions of the first wires and the first guide pipes are staggered by 180° in a circumferential direction and four of the second wires and the second guide pipes are inserted through the inside of the insertion portion in a condition in which the positions of the second wires and the second guide pipes are staggered by 90° in the circumferential direction so that, as a result, the first bending region is independently bendable in two directions by means of the first wires and the second bending region is bendable in four directions together with the first bending region by means of the second wires.

4. The endoscope according to claim 2, wherein two of the first wires and the first guide pipes are inserted through the inside of the insertion portion in a condition in which the positions of the first wires and the first guide pipes are staggered by 180° in a circumferential direction and four of the second wires and the second guide pipes are inserted through the inside of the insertion portion in a condition in which the positions of the second wires and the second guide pipes are staggered by 90° in the circumferential direction so that, as a result, the first bending region is independently bendable in two directions by means of the first wires and the second bending region is bendable in four directions together with the first bending region by means of the second wires.

5. The endoscope according to claim 1, wherein four of the first wires and the first guide pipes are inserted through the inside of the insertion portion in a condition in which the positions of the first wires and the first guide pipes are staggered by 90° in a circumferential direction and two of the second wires and the second guide pipes are inserted through the inside of the insertion portion in a condition in which the positions of the second wires and the second guide pipes are staggered by 180° in the circumferential direction so that, as a result, the first bending region is independently bendable in four directions by means of the first wires and the second bending region is bendable in two directions together with the first bending region by means of the second wires.

6. The endoscope according to claim 2, wherein four of the first wires and the first guide pipes are inserted through the inside of the insertion portion in a condition in which the positions of the first wires and the first guide pipes are staggered by 90° in a circumferential direction and two of the second wires and the second guide pipes are inserted through the inside of the insertion portion in a condition in which the positions of the second wires and the second guide pipes are staggered by 180° in the circumferential direction so that, as a result, the first bending region is independently bendable in four directions by means of the first wires and the second bending region is bendable in two directions together with the first bending region by means of the second wires.

7. The endoscope according to claim 1, wherein four of the first wires and the first guide pipes are inserted through the inside of the insertion portion in a condition in which the positions of the first wires and the first guide pipes are staggered by 90° in a circumferential direction and four of the second wires and the second guide pipes are inserted through the inside of the insertion portion in a condition in which the positions of the second wires and the second guide pipes are staggered by 90° in the circumferential direction so that, as a result, the first bending region is independently bendable in four directions by means of the first wires and the second bending region is bendable in four directions together with the first bending region by means of the second wires.

8. The endoscope according to claim 2, wherein four of the first wires and the first guide pipes are inserted through the inside of the insertion portion in a condition in which the positions of the first wires and the first guide pipes are staggered by 90° in a circumferential direction and four of the second wires and the second guide pipes are inserted through the inside of the insertion portion in a condition in which the positions of the second wires and the second guide pipes are staggered by 90° in the circumferential direction so that, as a result, the first bending region is independently bendable in four directions by means of the first wires and the second bending region is bendable in four directions together with the first bending region by means of the second wires.
